(19) United States
(12) Patent Application Publication
(10) Pub. No.: US 2024/0065639 A1
(43) Pub. Date: Feb. 29, 2024

(54) INFORMATION PROCESSING APPARATUS, INFORMATION PROCESSING METHOD, AND NON-TRANSITORY COMPUTER-READABLE STORAGE MEDIUM

(71) Applicant: NEC Corporation, Tokyo (JP)

(72) Inventor: Kazuya Kawakami, Tokyo (JP)

(73) Assignee: NEC Corporation, Tokyo (JP)

(21) Appl. No.: 18/236,141

(22) Filed: Aug. 21, 2023

(30) Foreign Application Priority Data
Aug. 25, 2022 (JP) 2022-133879

(51) Int. Cl. A61B 5/00; A61B 5/0205; A61B 5/16

(52) U.S. Cl. CPC A61B 5/74; A61B 5/02055; A61B 5/165; A61B 5/6802

(57) ABSTRACT

An information processing apparatus includes a biometric information processing unit that determines timing at which the biometric information satisfies a criterion by processing biometric information of a subject, an acquisition unit that acquires information indicating an environment of the subject at a date and time determined by using the timing, and an output unit that performs an output including the environment information.

This application is based upon and claims the benefit of priority from Japanese Patent Application No. 2022-133879 filed on Aug. 25, 2022, the disclosure of which is incorporated herein in its entirety by reference.

### TECHNICAL FIELD

The present invention relates to an information processing apparatus, a prediction apparatus, an information processing method, a prediction method, and a program.

### BACKGROUND ART

Biometric information such as an iris is used in various scenes. For example, Japanese Patent Application Publication No. 2001-128958 (PTL 1) describes that iris data are registered for each person, a person is determined by using the iris data, and also a health check for the person is performed.

Further, Japanese Patent Application Publication No. 2004-274519 (PTL 2) discloses an incoming call response system in which a transmitter's body condition is detected and incoming call response processing of a type suitable for the body condition is performed at a time of an incoming call from a transmission source. PTL 2 exemplifies, as one example of a body condition, information acquired by a camera photographing an iris.

Further, Japanese Patent Application Publication (Translation of PCT Application) No. 2004-517385 (PTL 3) describes that, in a vending machine selling a tea beverage, a user's iris is photographed, a user's health condition is decided by using the iris, and a medicine material and quantity thereof for the better health condition are determined and mixed in water.

Further, Japanese Patent Application Publication No. 2005-237561 (PTL 4) describes that user's mental and physical status is decided by using at least any of a degree of perspiration, a pulse, a respiratory pattern, a pupil diameter, and an iris pattern.

### SUMMARY

Biometric information of a person and environment information of the person are closely related to each other. Thus, environment information when biometric information satisfies a criterion is considered of value to the person. However, none of the above-described documents mentions environment information when biometric information satisfies a criterion.

In view of the above-described problem, an example object of the invention is to provide an information processing apparatus, a prediction apparatus, an information processing method, a prediction method, and a program for facilitating recognition of environment information when biometric information satisfies a criterion.

According to one aspect of the present invention, provided is an information processing apparatus including:

at least one memory configured to store instructions; and

at least one processor configured to execute the instructions to:
determine timing at which the biometric information satisfies a criterion by processing biometric information of a subject;
acquire environment information indicating an environment of the subject at a date and time determined by using the timing; and
perform an output including the environment information.

According to one aspect of the present invention, provided is a prediction apparatus including:

at least one memory configured to store instructions; and

at least one processor configured to execute the instructions to:
acquire subject identification information identifying a subject and environment information indicating an environment of a subject;
acquire, for each subject, from a storage unit that stores statistical data or a model for generating information relating to a possibility that biometric information of the subject satisfies a criterion in an environment indicated by the environment information and the subject identification information in association with each other, the statistical data or the model associated with the acquired subject identification information; and
generate, by using the acquired statistical data or the model acquired and the acquired environment information, prediction information relating to a possibility that biometric information of the subject satisfies a criterion in an environment indicated by the environment information.

According to one aspect of the present invention, provided is an information processing method including,

by a computer:

determining timing at which the biometric information satisfies a criterion by processing biometric information of a subject;

acquiring environment information indicating an environment of the subject at a date and time determined by using the timing; and

performing an output including the environment information.

According to one aspect of the present invention, provided is a prediction method including,

by a computer:

acquiring subject identification information identifying a subject and environment information indicating an environment of a subject;

acquiring, for each subject, from a storage unit that stores statistical data or a model for generating information relating to a possibility that biometric information of the subject satisfies a criterion in an environment indicated by the environment information and the subject identification information in association with each other, the statistical data or the model associated with the acquired subject identification information; and

generating, by using the statistical data or the model, and the environment information, prediction information relating to a possibility that biometric information of the subject satisfies a criterion in an environment indicated by the environment information.

According to one aspect of the present invention, provided is a non-transitory computer-readable storage medium storing a program for causing a computer to execute:

a process of determining timing at which the biometric information satisfies a criterion by processing biometric information of a subject;

a process of acquiring environment information indicating an environment of the subject at a date and time determined by using the timing; and

a process of performing an output including the environment information.

According to one aspect of the present invention, provided is a non-transitory computer-readable storage medium storing a program for causing a computer to execute:

a process of acquiring subject identification information identifying a subject and environment information indicating an environment of a subject;

a process of acquiring, for each subject, from a storage unit that stores statistical data or a model for generating information relating to a possibility that biometric information of the subject satisfies a criterion in an environment indicated by the environment information and the subject identification information in association with each other, the statistical data or the model associated with the acquired subject identification information; and

a process of generating, by using the acquired statistical data or the model and the acquired environment information, prediction information relating to a possibility that biometric information of the subject satisfies a criterion in an environment indicated by the environment information.

According to one aspect of the present invention, an information processing apparatus, a prediction apparatus, an information processing method, a prediction method, and a program for facilitating recognition of environment information when biometric information satisfies a criterion are acquired.

### BRIEF DESCRIPTION OF THE DRAWINGS

FIG. 1 is a diagram illustrating an overview of an information processing apparatus according to an example embodiment.

FIG. 2 is a diagram illustrating one example of a usage environment of the information processing apparatus.

FIG. 3 is a diagram illustrating a detailed example of a function configuration of the information processing apparatus.

FIG. 4 is a diagram for describing one example of information stored in a storage unit.

FIG. 5 is a diagram illustrating one example of a function configuration of a second terminal.

FIG. 6 is a diagram illustrating a hardware configuration example of the information processing apparatus.

FIG. 7 is a flowchart illustrating a first example of processing performed by the information processing apparatus.

FIG. 8 is a flowchart illustrating a second example of processing performed by the information processing apparatus.

FIG. 9 is a flowchart illustrating one example of processing performed by the second terminal.

### EXAMPLE EMBODIMENT

Hereinafter, an example embodiment of the present invention will be described by using drawings. Note that, in every drawing, a similar component is given a similar sign, and description thereof is not included as appropriate.

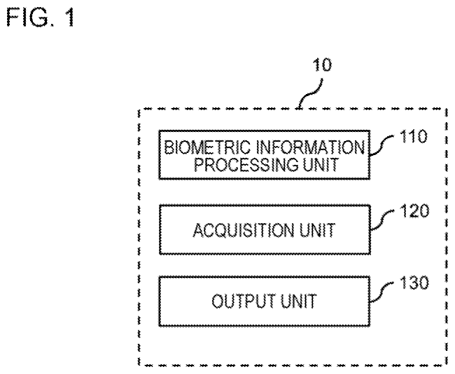

FIG. 1 is a diagram illustrating an overview of an information processing apparatus 10 according to an example embodiment. The information processing apparatus 10 includes a biometric information processing unit 110, an acquisition unit 120, and an output unit 130. The biometric information processing unit 110 determines timing at which the biometric information satisfies a criterion by processing biometric information of a subject. The acquisition unit 120 acquires information indicating an environment of a subject at a date and time determined by using the timing. Hereinafter, the information will be described as environment information. The environment information may indicate an environment of timing at which biometric information satisfies a criterion, may indicate an environment shortly before the timing, may indicate an environment shortly after the timing, or may indicate at least two environments thereof. A date and time of environment information is preferably set appropriately according to a purpose of use of the environment information. The output unit 130 performs an output including environment information.

According to the information processing apparatus 10, use of an output by the output unit 130 enables easy recognition of an environment of a subject at timing at which biometric information of the subject satisfies a criterion.

For example, when a time of biometric information satisfying a criterion is a time of a subject feeling stress, the timing means a possibility of immediate danger to the subject. In such a case, performing an output by the output unit 130 can notify another person of the possibility.

Further, when information output by the output unit 130, for example, environment information, is stored in a storage unit and the stored information is used as training data for machine learning or the information is statistically processed, an environment where there is a high possibility that biometric information of a subject satisfies a criterion, for example, an environment where there is a high possibility that the subject feels stress, can be determined.

Hereinafter, the information processing apparatus 10 will be described in detail. In the following description, one example of a criterion used by the biometric information processing unit 110 is a criterion for deciding that a subject feels stress.

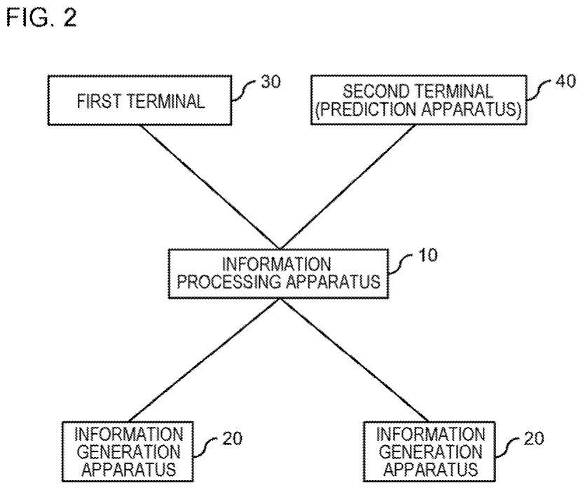

FIG. 2 is a diagram illustrating one example of a usage environment of the information processing apparatus 10. The information processing apparatus 10 is used together with an information generation apparatus 20, at least one first terminal 30, and at least one second terminal 40. The second terminal 40 is one example of a prediction apparatus.

The information generation apparatus 20 generates biometric information of a subject. As one example, the information generation apparatus 20 is a portable terminal, for example, a wearable device such as a smart glass or a smartwatch. In this case, the information generation apparatus 20 is prepared for each of a plurality of subjects. However, the information generation apparatus 20 may be used in common by a plurality of subjects. In this case, one example of the information generation apparatus 20 is a stationary terminal. The biometric information generated by the information generation apparatus 20 is, but not limited to, at least one of an iris, a heart rate, a body temperature, an amount of perspiration, a duration of sleep, a bedtime, and a wake-up time. Then, the information generation apparatus 20 transmits, to the information processing apparatus 10, the generated biometric information together with information capable of determining the subject, that is, subject determination information, and a generation date and time of the biometric information. A transmission timing thereof may be real time, or may be in batches.

Further, the information generation apparatus 20 generates at least a part of environment information. For example, the information generation apparatus 20 generates, as at least a part of environment information, at least one of position information indicating a position of a subject, voice information indicating a voice uttered around a subject, and an image including at least one of a subject and a periphery of a subject. When the information generation apparatus 20 includes a vibration sensor or an atmospheric temperature sensor, the environment information may include information indicating a vibration generated in a subject or an atmospheric temperature around a subject. Then, the information generation apparatus 20 transmits the generated environment information to the information processing apparatus 10. The information generation apparatus 20 may transmit the environment information together with biometric information, or may transmit the environment information to the information processing apparatus 10 in response to a request from the information processing apparatus 10.

When biometric information and subject determination information are acquired from the information generation apparatus 20, the information processing apparatus 10 processes the biometric information in real time as needed, and stores the biometric information in association with a generation date and time thereof and a subject. Then, when it can be seen that biometric information satisfies a criterion as a result of performing processing in real time, the information processing apparatus 10 decides that there is a possibility of immediate danger to a subject, and transmits information including environment information to the first terminal 30. In this case, the environment information includes at least one of voice information indicating a voice uttered around a subject and an image including at least one of a subject and a periphery of a subject. The environment information being transmitted to the first terminal 30 at this time includes at least one of environment information of timing at which biometric information satisfies a criterion, environment information a first reference period of time earlier than the timing, and environment information a second reference period of time later than the timing. Herein, both of the first reference period of time and the second reference period of time are, for example, but not limited to, equal to or more than one second and equal to or less than one minute. Further, the information processing apparatus 10 may transmit, to the first terminal 30, environment information of a whole period of time from the first reference period of time earlier than timing at which biometric information satisfies a criterion to the second reference period of time later than the timing.

The first terminal 30 is a terminal being operated by a police staff member or a security company staff member, and is a transmission destination of an output by the output unit 130 of the information processing apparatus 10 when there is a possibility of immediate danger to a subject. Thus, a person who operates the first terminal 30, for example, a police officer or a security officer, can objectively recognize that there is a possibility of immediate danger to the subject, by checking environment information, for example, an image or a voice, from the output unit 130. Note that, the first terminal 30 may be a portable terminal, or may be a stationary terminal.

Further, when timing at which biometric information satisfies a criterion is determined, the information processing apparatus 10 determines, based on the timing, which environment information generated at which date and time should be stored in a storage unit, and stores the determined environment information in the storage unit in association with the subject thereof. Then, the information processing apparatus 10 processes environment information stored in the storage unit for each subject, and thereby generates information capable of determining an environment where there is a high possibility that biometric information of the subject satisfies a criterion, for example, an environment where there is a high possibility that the subject feels stress. The information may be, for example, statistical data, may be a model generated by machine learning, or may be both thereof. The statistical data and the model are used by the second terminal 40.

The second terminal 40 is a terminal being operated by a subject. The second terminal 40 acquires statistical data or a model associated with the subject from the information processing apparatus 10, and generates, by using the statistical data or the model, information indicating whether there is a high possibility that biometric information of the subject satisfies a criterion, for example, whether there is a high possibility that the subject feels stress when an environment of the subject is in a particular state.

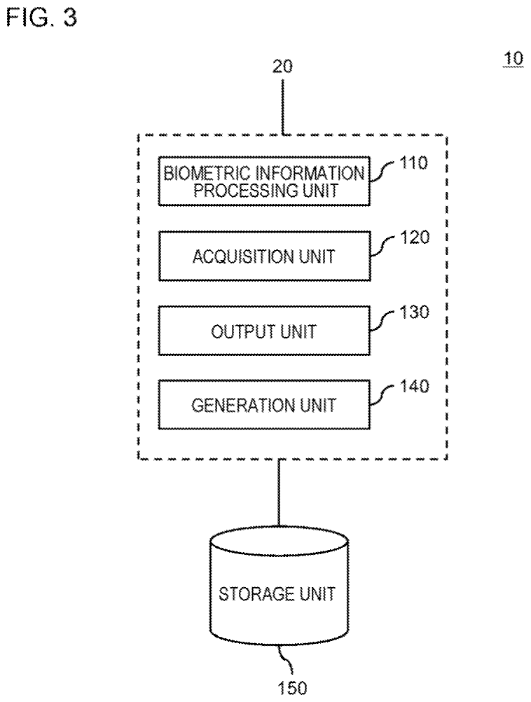

FIG. 3 is a diagram illustrating a detailed example of a function configuration of the information processing apparatus 10. The information processing apparatus 10 includes the biometric information processing unit 110, the acquisition unit 120, and the output unit 130 illustrated in FIG. 1, and also includes a generation unit 140. Further, the information processing apparatus 10 can use a storage unit 150. In the example illustrated in the present figure, the storage unit 150 is a part of the information processing apparatus 10. However, the storage unit 150 may be positioned outside the information processing apparatus 10.

The biometric information processing unit 110, by processing biometric information of a subject, determines timing at which the biometric information satisfies a criterion, for example, timing at which the subject feels stress, as described by using FIG. 1.

For example, when biometric information includes an iris, the biometric information processing unit 110 determines timing at which change in the iris satisfies a criterion. Further, when biometric information includes a heart rate or an amount of perspiration, the biometric information processing unit 110 determines timing at which the heart rate or the amount of perspiration becomes equal to or more than a reference value. Further, when biometric information includes a body temperature, the biometric information processing unit 110 determines timing at which an amount of change, for example, an amount of increase, in the body temperature becomes equal to or more than a reference value.

The above-described example is applicable to both a case of detecting that there is a possibility of immediate danger to a subject and a case of determining an environment where there is a high possibility that biometric information of a subject satisfies a criterion. On the other hand, as an example applicable to a latter example, the following can be further exemplified.

For example, when biometric information includes a duration of sleep, the biometric information processing unit 110 determines timing at which the duration of sleep becomes equal to or less than a reference value. Further, when biometric information includes a bedtime, the biometric information processing unit 110 determines timing at which the bedtime becomes later than a reference time, or timing at which a period of time from lying in a sleeping pose to actually going to sleep becomes longer than a reference period of time. Further, when biometric information includes a wake-up time, the biometric information processing unit 110 determines timing at which the wake-up time becomes earlier than a reference time.

The acquisition unit 120 determines, by using timing determined by the biometric information processing unit 110, which environment information generated at which date and time should be acquired, and acquires the determined environment information, as described by using FIG. 1.

For example, when there is a possibility of immediate danger to a subject, the acquisition unit 120 acquires at least one of environment information of timing at which biometric information satisfies a criterion and environment information a reference period of time earlier than the timing. At this time, the acquisition unit 120 may acquire environment information of a whole period of time from timing at which biometric information satisfies a criterion to a reference period of time later than the timing.

Further, when information capable of determining an environment where there is a high possibility that a subject feels stress is generated by the information processing apparatus 10, the acquisition unit 120 acquires at least one of environment information of timing at which biometric information satisfies a criterion, environment information a first reference period of time earlier than the timing, and environment information a second reference period of time later than the timing. At this time, the acquisition unit 120 may acquire environment information of a whole period of time from the first reference period of time earlier than timing at which biometric information satisfies a criterion to the second reference period of time later than timing at which biometric information satisfies a criterion.

Note that, after environment information that should be acquired is determined, the acquisition unit 120 may acquire the environment information from the information generation apparatus 20.

Further, environment information generated at a same timing as biometric information may sometimes be transmitted by the information generation apparatus 20 to the information processing apparatus 10 together with the biometric information. In this case, the biometric information processing unit 110 of the information processing apparatus 10 stores acquired environment information in the storage unit 150 in association with a generation timing thereof. Then, the acquisition unit 120 acquires environment information from the storage unit 150.

Further, environment information generated by the information generation apparatus 20 may sometimes be stored in an external storage. In this case, the acquisition unit 120 may acquire environment information from the storage.

The acquisition unit 120 acquires, as at least a part of environment information, at least one of position information indicating a position of a subject, voice information indicating a voice uttered around a subject, and an image including at least one of a subject and a periphery of a subject, from the information generation apparatus 20. Furthermore, the acquisition unit 120 may acquire, as a part of environment information, at least one of a time and a day of a week, from the information generation apparatus 20.

Further, the acquisition unit 120 may acquire a part of environment information from an apparatus other than the information generation apparatus 20. For example, the acquisition unit 120 may acquire schedule information from a schedule management apparatus storing schedule information of a subject. The schedule information includes a date and time, information indicating an action performed by a subject at the date and time, and information indicating a destination of visit and a purpose of visit. Further, the acquisition unit 120 may acquire weather information of a position of a subject from a weather information storage apparatus storing weather information. The weather information includes at least one of weather, wind strength, an amount of pollen, an amount of micro particulate matter such as PM2.5, an ultraviolet intensity, and an atmospheric pressure, but may further include other information.

The output unit 130 performs an output including environment information acquired by the acquisition unit 120, as described by using FIG. 1. For example, when there is a possibility of immediate danger to a subject, the output unit 130 performs the output to the first terminal 30 being a notification destination set in advance. In this case, the information generation apparatus 20 transmits biometric information to the information processing apparatus 10 in real time after the biometric information is generated. Further, the biometric information processing unit 110, the acquisition unit 120, and the output unit 130 of the information processing apparatus 10 perform processing in real time after the biometric information is acquired.

Further, the output unit 130 performs an output including environment information acquired by the acquisition unit 120, to the storage unit 150, and stores the environment information in the storage unit 150. A specific example of environment information in this case may be all of the above-described examples.

Note that, when environment information to be output includes position information of a subject, the output unit 130 may convert, by using map data, the position information into attribute data of the position. One example of the attribute data is, but not limited to, a type of a road, a type of a store, and a type of public transportation.

The generation unit 140 generates, by using environment information of each subject stored in the storage unit 150, at least one of statistical data and a model for generating information relating to a possibility that biometric information of a subject satisfies a criterion in a certain environment. For example, when the criterion is determined based on whether a subject feels stress, the statistical data or the model generates information relating to a possibility that a subject feels stress in a certain environment. The generation unit 140 stores the generated statistical data or the model in the storage unit 150 in association with a subject.

The statistical data generated by the generation unit 140 may indicate, for example, a result of totalizing, for each condition indicated by environment information, the number of times biometric information satisfies a criterion. Further, the model generated by the generation unit 140 outputs, for example, upon input of environment information, a numerical value indicating a possibility that a subject feels stress in the environment information. The model is generated, for example, by using machine learning such as deep learning.

The environment information used by the generation unit 140 includes at least one of position information indicating a position of a subject, voice information indicating a voice uttered around a subject, an image including at least one of a subject and a periphery of a subject, a time, a day of a week, weather information, and schedule information.

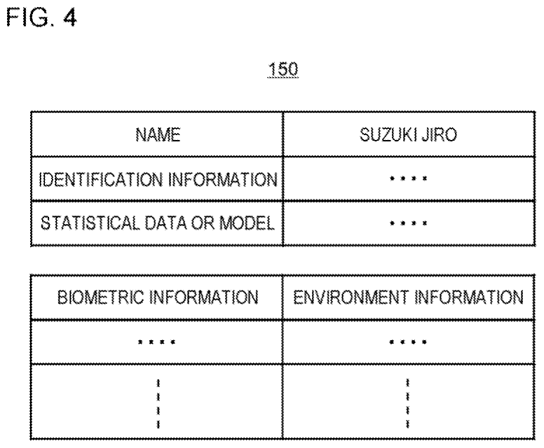

FIG. 4 is a diagram for describing one example of information stored in the storage unit 150. The storage unit 150 stores, for each subject, a name of a subject, identification information, statistical data or a model generated by the generation unit 140, biometric information when a criterion is satisfied, and environment information acquired by the acquisition unit 120 in relation to the biometric information. The environment information and the biometric information are stored in association with each other. Note that, the storage unit 150 may store all pieces of biometric information and environment information generated by the information generation apparatus 20 in association with timing at which the biometric information and the environment information are generated.

Herein, the identification information may be any information, as long as the information is capable of identifying a subject. As one example, the identification information may be biometric information such as face information or iris information, or may be an alphanumeric string given for each subject, a so-called ID.

Further, although not illustrated, the storage unit 150 stores information determining the first terminal 30. The information is used when the information processing apparatus 10 transmits information to the first terminal 30.

Note that, processing performed by the biometric information processing unit 110 may be performed by the information generation apparatus 20. In this case, the information generation apparatus 20 transmits, to the information processing apparatus 10, environment information when biometric information satisfies a criterion together with the biometric information. The acquisition unit 120 of the information processing apparatus 10 acquires the environment information and the biometric information, and performs processing.

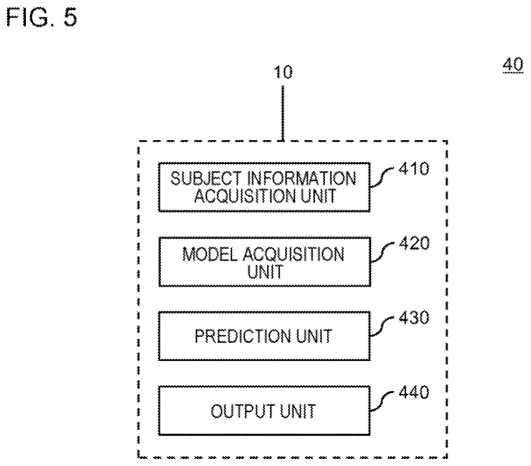

FIG. 5 is a diagram illustrating one example of a function configuration of the second terminal 40. The second terminal 40 includes a subject information acquisition unit 410, a model acquisition unit 420, a prediction unit 430, and an output unit 440. The second terminal 40 generates, by using statistical data or a model generated by the information processing apparatus 10, information relating to a possibility that biometric information of the subject satisfies a criterion, for example, information relating to a possibility that the subject feels stress when an environment of the subject is in a particular state, as described by using FIG. 2. Hereinafter, the information will be described as prediction information. One example of the prediction information indicates whether there is a high possibility that biometric information of the subject satisfies a criterion, for example, whether there is a high possibility that the subject feels stress. The prediction information may be a numerical value indicating the possibilities.

The subject information acquisition unit 410 acquires subject identification information identifying a subject and environment information indicating an environment of a subject. The subject identification information is used upon determining statistical data or a model. Further, the environment information acquired by the subject information acquisition unit 410 indicates an environment that may occur to the subject in future. Examples of the environment are, for example, as follows.

### First Example

The example is a case in which a subject desires to preliminarily estimate a possibility that biometric information satisfies a criterion upon traveling to a destination, for example, a possibility that the subject feels stress. In this case, environment information includes a departure place, a destination, and at least one of an estimated time of departure and an estimated time of arrival.

In the example, the subject information acquisition unit 410 may use schedule data of a subject upon acquiring environment information. In this case, the subject information acquisition unit 410 determines, based on the schedule data, a departure place, a destination, and at least one of an estimated time of departure and an estimated time of arrival. In this case, prediction information to be described later indicates a place and a time at which there is a possibility that a subject feels stress in a schedule of the subject.

### Second Example

The example is used when a subject determines a destination of moving house. A behavioral pattern of a person is often determined to some extent. A person often has a routine of what facility, for example, what store to visit at around what time, for example, on a day off and a holiday. This example assumes that a subject has moved to a candidate place for the destination of moving house, estimates a behavioral pattern when the routine is applied, and preliminarily estimates a possibility that biometric information of the subject satisfies a criterion, for example, a possibility that the subject feels stress in the behavioral pattern.

In the example, environment information includes region specification information specifying a region and behavioral pattern information indicating a behavioral pattern of a subject. The region indicated by the region specification information may be a unit of municipalities, or may be a smaller unit. The behavioral pattern information includes, for example, attribute information of a place where a subject visits and a time zone when the subject visits the place. The attribute information of a place is, for example, a type of a store or a facility. As one example, the attribute information of a place is, but not limited to, a grocery store or a fitness gym. Note that, when there are a plurality of places where a subject visits, a combination of attribute information of a place and a time zone is set for each of the plurality of places.

The model acquisition unit 420 acquires, from the information processing apparatus 10, statistical data or a model associated with subject identification information acquired by the subject information acquisition unit 410.

The prediction unit 430 generates, by using statistical data or a model acquired by the model acquisition unit 420 and environment information acquired by the subject information acquisition unit 410, information relating to a possibility that biometric information of a subject satisfies a criterion, that is, the above-described prediction information, in an environment indicated by the environment information acquired by the subject information acquisition unit 410.

For example, a case in which environment information is the above-described first example and the model acquisition unit 420 has acquired statistical data will be considered. In this case, the statistical data indicate, for example, the number of samples of each combination of an attribute of a place and a time. The statistical data may further include an item other than the environment information, for example, information relating to weather. On the other hand, the prediction unit 430 estimates a travel path by using a departure place and a destination. Then, the prediction unit 430 decides, for each spot in the travel path, an attribute of the spot and an estimated time for the spot, and determines whether a combination matching or similar to a combination with a large number of samples in the statistical data acquired by the model acquisition unit 420 is included among combinations of the attribute and the estimated time. Then, when the combination is included, the prediction unit 430 includes, in prediction information, the combination, that is, a particular spot included in the travel path and an estimated time of arrival in the spot. Note that, when the statistical data include information relating to weather, the prediction unit 430 may further use weather forecast in the travel path upon generating prediction information.

Further, a case in which environment information is the above-described first example and the model acquisition unit 420 has acquired a model will be considered. In this case, the model receives a combination of an attribute of a place and a time as an input, and outputs a score indicating a possibility that biometric information satisfies a criterion, for example, a score indicating a possibility that a subject feels stress. Then, the prediction unit 430 inputs, for each spot in a travel path, an attribute of the spot and an estimated time of arrival in the spot to the model, and causes the model to compute a score for each spot. Then, when there is a spot for which the score is equal to or more than a reference value, the spot and the estimated time of arrival in the spot are included in prediction information.

Further, a case in which environment information is the above-described second example will be considered. In this case as well, statistical data indicate, for example, the number of samples of each combination of an attribute of a place and a time. On the other hand, the prediction unit 430 selects, from map information, a place, for example, a store, associated with attribute information included in a behavioral pattern, in a region indicated by region specification information. Then, the prediction unit 430 assumes visiting the store in a time zone indicated by a behavioral pattern, and generates a behavioral pattern associated with region specification information. The behavioral pattern can be handled as data similar to a travel path in the first example. Thus, processing thereafter is similar to the case of the first example. Then, at least a part of prediction information generated in the example includes information relating to stress felt by a subject on a travel path to visit the place associated with the attribute information in the time zone indicated by the behavioral pattern.

The output unit 440 outputs prediction information generated by the prediction unit 430. For example, the output unit 440 causes a display included in the second terminal 40 to display prediction information. The output unit 440 may display, for example, an assumed travel path of a subject by means of a map or the like, and may display a spot of the assumed travel path included in prediction information in a different form (for example, by a different color) from another spot.

For example, when environment information is the first example, the output unit 440 may output, in addition to prediction information, recommendation information indicating how to make a possibility that biometric information satisfies a criterion lowered, for example, a subject feels less stress. One example of the recommendation information is a recommended time for at least one of a departure time and an estimated time of arrival. The recommended time indicates a time at which there is a low possibility that biometric information satisfies a criterion.

Note that, the output unit 440 may output statistical data per se. For example, the output unit 440 may output, as statistical data, a condition (for example, a weather condition, a place, a time zone, or the like) in which there is a high possibility that a subject feels stress. In this way, the subject can recognize an environment where he/she feels more stress.

Further, in the above-described first example, a server may include the subject information acquisition unit 410, the model acquisition unit 420, the prediction unit 430, and the output unit 440, instead of the second terminal 40. In this case, the subject information acquisition unit 410 acquires subject determination information from the second terminal 40, and generates environment information by using an apparatus storing schedule data. Then, the output unit 440 transmits prediction information to the second terminal 40. The second terminal 40 displays the prediction information and causes a subject to recognize the prediction information. Thereby, a subject can preliminarily recognize a place and a time at which there is a possibility that the subject feels stress in a schedule.

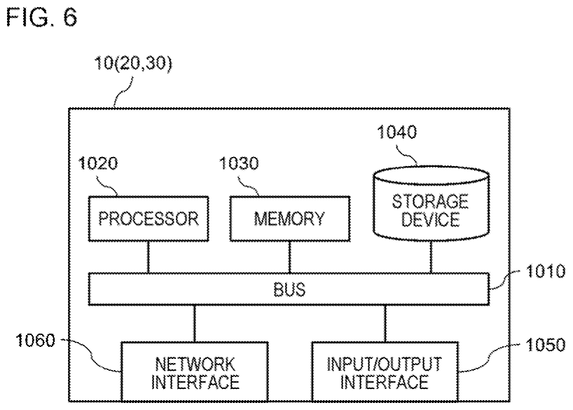

FIG. 6 is a diagram illustrating a hardware configuration example of the information processing apparatus 10. The information processing apparatus 10 includes a bus 1010, a processor 1020, a memory 1030, a storage device 1040, an input/output interface 1050, and a network interface 1060.

The bus 1010 is a data transmission path through which the processor 1020, the memory 1030, the storage device 1040, the input/output interface 1050, and the network interface 1060 transmit and receive data to and from one another. However, a method of connecting the processor 1020 and the like with one another is not limited to bus connection.

The processor 1020 is a processor achieved by a central processing unit (CPU), a graphics processing unit (GPU), or the like.

The memory 1030 is a main storage apparatus achieved by a random access memory (RAM) or the like.

The storage device 1040 is an auxiliary storage apparatus achieved by a hard disk drive (HDD), a solid state drive (SSD), a removable medium such as a memory card, a read only memory (ROM), or the like, and includes a storage medium. The storage medium of the storage device 1040 stores a program module for achieving each function (for example, the biometric information processing unit 110, the acquisition unit 120, the output unit 130, and the generation unit 140) of the information processing apparatus 10. Each of the program modules is read into the memory 1030 and executed by the processor 1020, and thereby each function unit associated with the program module is achieved. Further, the storage device 1040 also functions as the storage unit 150.

The input/output interface 1050 is an interface for connecting the information processing apparatus 10 with various kinds of input/output equipment.

The network interface 1060 is an interface for connecting the information processing apparatus 10 to a network. The network is, for example, a local area network (LAN) or a wide area network (WAN). A method by which the network interface 1060 connects to a network may be wireless connection, or may be wired connection. The information processing apparatus 10 may communicate with the information generation apparatus 20, the first terminal 30, and the second terminal 40 via the network interface 1060.

Note that, a hardware configuration of the information generation apparatus 20, the first terminal 30, and the second terminal 40 is also similar to the hardware configuration of the information processing apparatus 10 illustrated in FIG. 6.

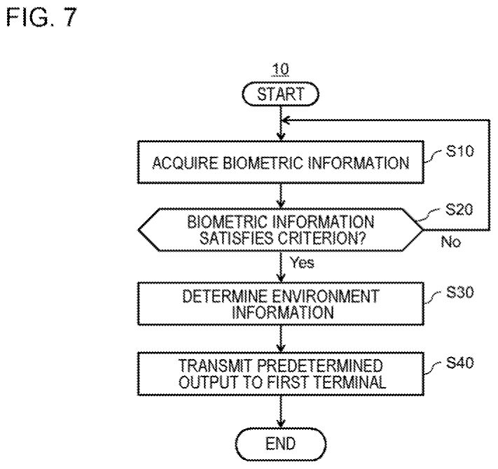

FIG. 7 is a flowchart illustrating a first example of processing performed by the information processing apparatus 10. In the example illustrated in the present figure, the information processing apparatus 10 processes biometric information generated by the information generation apparatus 20 in real time, and decides whether there is a possibility of immediate danger to a subject.

The information generation apparatus 20 repeatedly generates biometric information of a subject, and repeatedly generates, as at least a part of environment information, at least one of position information indicating a position of the subject, voice information indicating a voice uttered around the subject, and an image including at least one of the subject and a periphery of the subject. Then, when biometric information is generated, the information generation apparatus 20 transmits the generated biometric information to the information processing apparatus 10 in real time. The biometric information processing unit 110 of the information processing apparatus 10 acquires the biometric information (Step S10). At this time, the information generation apparatus 20 may transmit environment information together with the biometric information. In this case, the biometric information processing unit 110 also acquires environment information, and stores the acquired environment information in the storage unit 150.

When the biometric information processing unit 110 acquires biometric information, the acquisition unit 120 of the information processing apparatus 10 decides whether the biometric information satisfies a criterion in real time. When a criterion is not satisfied (Step S20: No), the information processing apparatus 10 returns to Step S10. On the other hand, when a criterion is satisfied (Step S20: Yes), the acquisition unit 120 acquires environment information generated by the information generation apparatus 20. A specific example of a generation timing of the environment information acquired herein is as described by using FIG. 3. The acquisition unit 120 may acquire the environment information from, for example, the storage unit 150, may acquire the environment information from the information generation apparatus 20, or may acquire the environment information from an apparatus different from the information generation apparatus 20 (Step S30).

Then, the output unit 130 of the information processing apparatus 10 transmits a predetermined output to the first terminal 30 (Step S40). The output includes the environment information acquired in Step S30. The environment information includes at least one of, or preferably all of position information indicating a position of the subject, voice information indicating a voice uttered around the subject, and an image including at least one of the subject and a periphery of the subject. Thus, a person who operates the first terminal 30 can objectively recognize that there is a possibility of immediate danger to the subject, by checking the environment information. Further, when the environment information includes position information of the subject, for example, a police officer or a security officer can immediately go to an actual place.

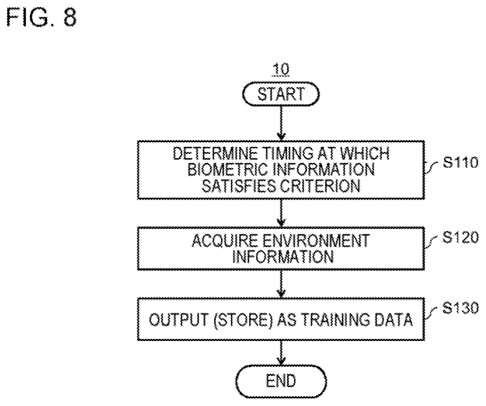

FIG. 8 is a flowchart illustrating a second example of processing performed by the information processing apparatus 10. The information processing apparatus 10 generates statistical data or a model for generating information relating to a possibility that biometric information of a subject satisfies a criterion in a certain environment, as described by using FIG. 3 and the like. In the example illustrated in the present figure, the information processing apparatus 10 prepares data for generating the statistical data or the model. The information processing apparatus 10 performs processing illustrated in the present figure for each subject.

In the example illustrated in the present figure, the biometric information processing unit 110 of the information processing apparatus 10 acquires, from the information generation apparatus 20, all pieces of biometric information and environment information generated by the information generation apparatus 20. Then, the biometric information processing unit 110 stores the biometric information and the environment information in the storage unit 150 in association with timing at which the biometric information and the environment information are generated, for example, a date and time. Then, the processing illustrated in the present figure is performed, for example, in batches.

The biometric information processing unit 110 of the information processing apparatus 10 determines biometric information satisfying a criterion among pieces of biometric information stored in the storage unit 150, and determines timing at which the biometric information is generated (Step S110). Next, the acquisition unit 120 of the information processing apparatus 10 determines, by using the timing determined in Step S110, a generation timing of environment information that should be data for generating the above-described statistical data or the model, and reads out environment information associated with the timing from the storage unit 150 (Step S120). Then, the output unit 130 of the information processing apparatus 10 stores the environment information in the storage unit 150 in association with a subject, as data for generating the above-described statistical data or the model (Step S130). At this time, the output unit 130 may also store, as reference information, the biometric information determined in Step S110.

Thereafter, the information processing apparatus 10 generates, at a necessary timing, the above-described statistical data or the model by processing the data stored in the storage unit 150 in Step S130, and stores the generated statistical data or the model in the storage unit 150 in association with a subject.

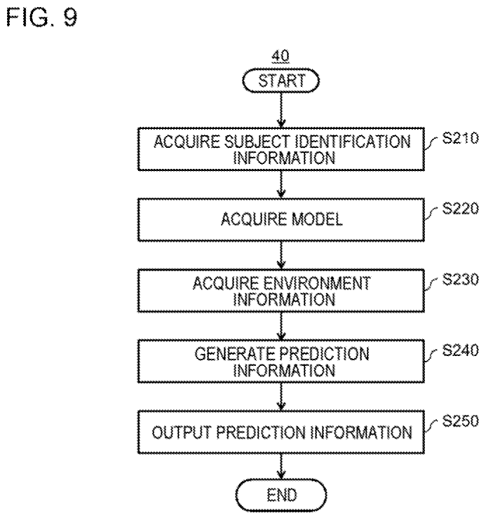

FIG. 9 is a flowchart illustrating one example of processing performed by the second terminal 40. In the present figure, the second terminal 40 generates, by using statistical data or a model generated by the information processing apparatus 10, prediction information, that is, information relating to a possibility that biometric information of a subject satisfies a criterion in a specified environment.

First, the subject information acquisition unit 410 of the second terminal 40 acquires subject identification information. For example, a subject inputs subject identification information of the subject to the second terminal 40 (Step S210).

Next, the model acquisition unit 420 of the second terminal 40 transmits the subject identification information to the information processing apparatus 10. The output unit 130 of the information processing apparatus 10 reads out statistical data or a model associated with the subject identification information from the storage unit 150, and transmits the statistical data or the model to the second terminal 40. The model acquisition unit 420 of the second terminal 40 acquires the statistical data or the model (Step S220).

Next, the subject information acquisition unit 410 of the second terminal 40 acquires environment information indicating an environment that may occur to the subject in future. A specific example of the environment information is, but not limited to, the first example and the second example described by using FIG. 5 (Step S230). Next, the prediction unit 430 of the second terminal 40 generates prediction information by using the statistical data or the model acquired in Step S220 and the environment information acquired in Step S230. A specific example of a method of generating the prediction information is as described by using FIG. 5 (Step S240). Thereafter, the output unit 440 of the second terminal 40 outputs the prediction information (Step S250).

According to the present example embodiment described above, when biometric information of a subject satisfies a criterion, the output unit 130 outputs environment information of the subject at a date and time determined by using timing at which the criterion is satisfied. Accordingly, use of an output by the output unit 130 enables easy recognition of an environment of a subject at timing at which biometric information of the subject satisfies a criterion.

For example, when the output unit 130 performs an output to a notification destination set in advance, for example, a terminal being operated by a police staff member or a security company staff member, the notification destination can recognize environment information of a subject. In this case, since environment information includes at least one of position information indicating a position of a subject, voice information indicating a voice uttered around the subject, and an image including at least one of the subject and a periphery of the subject, the notification destination can objectively recognize an environment of a subject.

Further, the generation unit 140 generates at least one of statistical data being statistically processed environment information output by the output unit 130 and a model using the environment information as training data. Then, the second terminal 40 generates, by using the statistical data or the model, information relating to a possibility that biometric information of a subject satisfies a criterion in a specified environment, for example, a possibility that a subject feels stress. Accordingly, a user of the second terminal 40, for example, a subject, can recognize a possibility that biometric information of the subject satisfies a criterion in a specified environment.

While the example embodiment of the present invention has been described above with reference to the drawings, the example embodiment is an exemplification of the present invention, and various configurations other than the above can be employed.

While the invention has been particularly shown and described with reference to example embodiments thereof, the invention is not limited to these embodiments. It will be understood by those of ordinary skill in the art that various changes in form and details may be made therein without departing from the spirit and scope of the present invention as defined by the claims.

Further, while a plurality of processes (pieces of processing) are described in order in a plurality of flowcharts used in the above description, execution order of processes executed in each example embodiment is not limited to the described order. The order of the illustrated processes can be changed in each example embodiment, as long as the change does not detract from contents. Further, the above example embodiments can be combined, as long as contents do not contradict each other.

The whole or part of the example embodiments disclosed above can be described as, but not limited to, the following supplementary notes.
1. An information processing apparatus including:
   a biometric information processing unit that determines timing at which the biometric information satisfies a criterion by processing biometric information of a subject;
   an acquisition unit that acquires environment information indicating an environment of the subject at a date and time determined by using the timing; and
   an output unit that performs an output including the environment information.
2. The information processing apparatus according to supplementary note 1, wherein
   the criterion is a criterion for deciding that the subject feels stress.
3. The information processing apparatus according to supplementary note 1 or 2, wherein
   the output unit performs the output to a notification destination set in advance.
4. The information processing apparatus according to supplementary note 3, wherein
   the notification destination includes a terminal being operated by a police staff member or a security company staff member.
5. The information processing apparatus according to supplementary note 3 or 4, wherein
   the environment information includes at least one of position information indicating a position of the subject, voice information indicating a voice uttered around the subject, and an image including at least one of the subject and a periphery of the subject.
6. The information processing apparatus according to any one of supplementary notes 3 to 5, wherein
   the biometric information processing unit, the acquisition unit, and the output unit perform processing in real time after the biometric information is generated.
7. The information processing apparatus according to any one of supplementary notes 3 to 6, wherein
   the biometric information includes at least one of an iris, a heart rate, a body temperature, and an amount of perspiration.
8. The information processing apparatus according to supplementary note 2, further including
   a generation unit that generates, by using the environment information included in the output, at least one of statistical data and a model for generating information relating to a possibility that biometric information of the subject satisfies a criterion in a certain environment.
9. The information processing apparatus according to supplementary note 8, wherein
   the environment information includes at least one of position information indicating a position of the subject, voice information indicating a voice uttered around the subject, an image including at least one of the subject and a periphery of the subject, a time, a day of a week, weather information, and schedule information.
10. The information processing apparatus according to supplementary note 8 or 9, wherein
   the biometric information includes at least one of an iris, a heart rate, a body temperature, an amount of perspiration, a duration of sleep, a bedtime, and a wake-up time.
11. The information processing apparatus according to any one of supplementary notes 1 to 10, wherein
   the biometric information is acquired by an apparatus worn by the subject.
12. A prediction apparatus including:
   a subject information acquisition unit that acquires subject identification information identifying a subject and environment information indicating an environment of a subject;
   a model acquisition unit that acquires, for each subject, from a storage unit that stores statistical data or a model for generating information relating to a possibility that biometric information of the subject satisfies a criterion in an environment indicated by the environment information and the subject identification information in association with each other, the statistical data or the model associated with the subject identification information acquired by the subject information acquisition unit; and
   a prediction unit that generates, by using the statistical data or the model acquired by the model acquisition unit and the environment information acquired by the subject information acquisition unit, prediction information relating to a possibility that biometric information of the subject satisfies a criterion in an environment indicated by the environment information.
13. The prediction apparatus according to supplementary note 12, wherein
   the subject information acquisition unit acquires, as the environment information, a departure place, a destination, and at least one of an estimated time of departure and an estimated time of arrival, and
   the prediction information relates to stress felt by the subject upon traveling to the destination.
14. The prediction apparatus according to supplementary note 12, wherein
   the subject information acquisition unit acquires, as the environment information, region specification information specifying a region and behavioral pattern information indicating a behavioral pattern of the subject, and
   the prediction information relates to stress felt by the subject when the subject takes the behavioral pattern in the region indicated by the region specification information.
15. The prediction apparatus according to supplementary note 14, wherein
   the behavioral pattern information includes attribute information of a place where the subject visits and a time zone when the subject visits the place, and
   the prediction unit
      determines, by using the region specification information and attribute information of the place, a place where the subject stops by in the time zone in the region indicated by the region specification information, and
      generates, as at least a part of the prediction information, information relating to stress felt by the subject on a travel path to visit the determined place in the time zone.
16. An information processing method including,
   by a computer:
   determining timing at which the biometric information satisfies a criterion by processing biometric information of a subject;
   acquiring environment information indicating an environment of the subject at a date and time determined by using the timing; and performing an output including the environment information.
17. The information processing method according to supplementary note 16, wherein
   the criterion is a criterion for deciding that the subject feels stress.
18. The information processing method according to supplementary note 16 or 17, wherein
   the computer performs the output to a notification destination set in advance.
19. The information processing method according to supplementary note 18, wherein
   the notification destination includes a terminal being operated by a police staff member or a security company staff member.
20. The information processing method according to supplementary note 18 or 19, wherein
   the environment information includes at least one of position information indicating a position of the subject, voice information indicating a voice uttered around the subject, and an image including at least one of the subject and a periphery of the subject.
21. The information processing method according to any one of supplementary notes 18 to 20, wherein
   the computer performs processing in real time after the biometric information is generated.
22. The information processing method according to any one of supplementary notes 18 to 21, wherein
   the biometric information includes at least one of an iris, a heart rate, a body temperature, and an amount of perspiration.
23. The information processing method according to supplementary note 17, wherein
   the computer generates, by using the environment information included in the output, at least one of statistical data and a model for generating information relating to a possibility that biometric information of the subject satisfies a criterion in a certain environment.
24. The information processing method according to supplementary note 23, wherein
   the environment information includes at least one of position information indicating a position of the subject, voice information indicating a voice uttered around the subject, an image including at least one of the subject and a periphery of the subject, a time, a day of a week, weather information, and schedule information.
25. The information processing method according to supplementary note 23 or 24, wherein
   the biometric information includes at least one of an iris, a heart rate, a body temperature, an amount of perspiration, a duration of sleep, a bedtime, and a wake-up time.
26. The information processing method according to any one of supplementary notes 16 to 25, wherein
   the biometric information is acquired by an apparatus worn by the subject.
27. A prediction method including,
   by a computer:
   acquiring subject identification information identifying a subject and environment information indicating an environment of a subject;
   acquiring, for each subject, from a storage unit that stores statistical data or a model for generating information relating to a possibility that biometric information of the subject satisfies a criterion in an environment indicated by the environment information and the subject identification information in association with each other, the statistical data or the model associated with the acquired subject identification information; and
   generating, by using the statistical data or the model and the environment information, prediction information relating to a possibility that biometric information of the subject satisfies a criterion in an environment indicated by the environment information.
28. The prediction method according to supplementary note 27, wherein
   the computer acquires, as the environment information, a departure place, a destination, and at least one of an estimated time of departure and an estimated time of arrival, and
   the prediction information relates to stress felt by the subject upon traveling to the destination.
29. The prediction method according to supplementary note 27, wherein
   the computer acquires, as the environment information, region specification information specifying a region and behavioral pattern information indicating a behavioral pattern of the subject, and
   the prediction information relates to stress felt by the subject when the subject takes the behavioral pattern in the region indicated by the region specification information.
30. The prediction method according to supplementary note 29, wherein
   the behavioral pattern information includes attribute information of a place where the subject visits and a time zone when the subject visits the place, and
   the computer
      determines, by using the region specification information and attribute information of the place, a place where the subject stops by in the time zone in the region indicated by the region specification information, and
      generates, as at least a part of the prediction information, information relating to stress felt by the subject on a travel path to visit the determined place in the time zone.
31. A program for causing a computer to include:
   a biometric information processing unit that determines timing at which the biometric information satisfies a criterion by processing biometric information of a subject;
   an acquisition unit that acquires environment information indicating an environment of the subject at a date and time determined by using the timing; and
   an output unit that performs an output including the environment information.
32. The program according to supplementary note 31, wherein
   the criterion is a criterion for deciding that the subject feels stress.
33. The program according to supplementary note 31 or 32, further causing the computer to perform the output to a notification destination set in advance.
34. The program according to supplementary note 33, wherein
   the notification destination includes a terminal being operated by a police staff member or a security company staff member.
35. The program according to supplementary note 33 or 34, wherein
   the environment information includes at least one of position information indicating a position of the subject, voice information indicating a voice uttered around the subject, and an image including at least one of the subject and a periphery of the subject.
36. The program according to any one of supplementary notes 33 to 35, further causing the computer to perform processing in real time after the biometric information is generated.
37. The program according to any one of supplementary notes 33 to 36, wherein
   the biometric information includes at least one of an iris, a heart rate, a body temperature, and an amount of perspiration.
38. The program according to supplementary note 32, further causing the computer to generate, by using the environment information included in the output, at least one of statistical data and a model for generating information relating to a possibility that biometric information of the subject satisfies a criterion in a certain environment.
39. The program according to supplementary note 38, wherein
   the environment information includes at least one of position information indicating a position of the subject, voice information indicating a voice uttered around the subject, an image including at least one of the subject and a periphery of the subject, a time, a day of a week, weather information, and schedule information.
40. The program according to supplementary note 38 or 39, wherein
   the biometric information includes at least one of an iris, a heart rate, a body temperature, an amount of perspiration, a duration of sleep, a bedtime, and a wake-up time.
41. The program according to any one of supplementary notes 31 to 40, wherein
   the biometric information is acquired by an apparatus worn by the subject.
42. A program for causing a computer to include:
   a subject information acquisition unit that acquires subject identification information identifying a subject and environment information indicating an environment of a subject;
   a model acquisition unit that acquires, for each subject, from a storage unit that stores statistical data or a model for generating information relating to a possibility that biometric information of the subject satisfies a criterion in an environment indicated by the environment information and the subject identification information in association with each other, the statistical data or the model associated with the subject identification information acquired by the subject information acquisition unit; and
   a prediction unit that generates, by using the statistical data or the model acquired by the model acquisition unit and the environment information acquired by the subject information acquisition unit, prediction information relating to a possibility that biometric information of the subject satisfies a criterion in an environment indicated by the environment information.
43. The program according to supplementary note 42, further causing the computer to acquire, as the environment information, a departure place, a destination, and at least one of an estimated time of departure and an estimated time of arrival, wherein
   the prediction information relates to stress felt by the subject upon traveling to the destination.
44. The program according to supplementary note 42, further causing the computer to acquire, as the environment information, region specification information specifying a region and behavioral pattern information indicating a behavioral pattern of the subject, wherein
   the prediction information relates to stress felt by the subject when the subject takes the behavioral pattern in the region indicated by the region specification information.
45. The program according to supplementary note 44, wherein
   the behavioral pattern information includes attribute information of a place where the subject visits and a time zone when the subject visits the place, the program further causing the computer to
      determine, by using the region specification information and attribute information of the place, a place where the subject stops by in the time zone in the region indicated by the region specification information, and
      generate, as at least a part of the prediction information, information relating to stress felt by the subject on a travel path to visit the determined place in the time zone.
46. A recording medium on which the program according to any one of supplementary notes 31 to 45 is recorded.

What is claimed is:

1. An information processing apparatus comprising:
at least one memory configured to store instructions; and
at least one processor configured to execute the instructions to:
determine timing at which the biometric information satisfies a criterion by processing biometric information of a subject;
acquire environment information indicating an environment of the subject at a date and time determined by using the timing; and
perform an output including the environment information.

2. The information processing apparatus according to claim 1, wherein
the criterion is a criterion for deciding that the subject feels stress.

3. The information processing apparatus according to claim 1, wherein
the at least one processor is further configured to execute the instructions to perform the output to a notification destination set in advance.

4. The information processing apparatus according to claim 2, wherein
the at least one processor is further configured to execute the instructions to generate, by using the environment information included in the output, at least one of statistical data and a model for generating information relating to a possibility that biometric information of the subject satisfies a criterion in a certain environment.

5. The information processing apparatus according to claim 1, wherein
the biometric information is acquired by an apparatus worn by the subject.

6. An information processing method comprising,
by a computer:
determining timing at which the biometric information satisfies a criterion by processing biometric information of a subject;
acquiring environment information indicating an environment of the subject at a date and time determined by using the timing; and
performing an output including the environment information.

7. The information processing method according to claim 6, wherein
the criterion is a criterion for deciding that the subject feels stress.

8. The information processing method according to claim 6, further comprising, by the computer, performing the output to a notification destination set in advance.

9. The information processing method according to claim 7, further comprising, by the computer, generating, by using the environment information included in the output, at least one of statistical data and a model for generating information relating to a possibility that biometric information of the subject satisfies a criterion in a certain environment.

10. The information processing method according to claim 6, wherein
the biometric information is acquired by an apparatus worn by the subject.

11. A non-transitory computer-readable storage medium storing a program for causing a computer to execute:
a process of determining timing at which the biometric information satisfies a criterion by processing biometric information of a subject;
a process of acquiring environment information indicating an environment of the subject at a date and time determined by using the timing; and
a process of performing an output including the environment information.

12. The non-transitory computer-readable storage medium according to claim 11, wherein
the criterion is a criterion for deciding that the subject feels stress.

13. The non-transitory computer-readable storage medium according to claim 11, wherein
the program causes the computer to further execute a process of performing the output to a notification destination set in advance.

14. The non-transitory computer-readable storage medium according to claim 12, wherein
the program causes the computer to further execute a process of generating, by using the environment information included in the output, at least one of statistical data and a model for generating information relating to a possibility that biometric information of the subject satisfies a criterion in a certain environment.

15. The non-transitory computer-readable storage medium s according to claim 11, wherein
the biometric information is acquired by an apparatus worn by the subject.
